(19) United States
(12) Patent Application Publication
(10) Pub. No.: US 2024/0321451 A1
(43) Pub. Date: Sep. 26, 2024

(54) System And Method For Diagnostic Coding

(71) Applicant: Bruce Wayne FallHowe, Colorado Springs, CO (US)

(72) Inventor: Bruce Wayne FallHowe, Colorado Springs, CO (US)

(21) Appl. No.: 18/428,802

(22) Filed: Jan. 31, 2024

Related U.S. Application Data
(63) Continuation-in-part of application No. 18/123,724, filed on Mar. 20, 2023.

(51) Int. Cl. G16H 50/20; G16H 70/20

(52) U.S. Cl. CPC G16H 50/20; G16H 70/20

(57) ABSTRACT

A computer implemented coding system for mandating correct medical diagnostic coding by a provider, comprises program code executable to receive patient encounter medical data associated with a patient. Medical elements performed during the patient encounter are compared and matched with up-to-date guidelines that include medical guideline, derived from a stored database of diagnostic code requirements determined via a deep machine learning/artificial intelligence acquisition of up-to-date medial data, by which a diagnosis is made and published for consideration by a treating provider. The matched guideline is published on a display screen visible to the treating provider where it is accepted or refused in favor of the treating providers alternative diagnosis. If accepted, an insurance code is determined and submitted for payment. If not, a list of missing medical procedures associated with the provider's diagnosis is determined and ordered before an insurance code may be assigned.

### REFERENCE TO RELATED APPLICATIONS

This application is a Continuation-in-Part and claims the priority of U.S. Ser. No. 18/123,724 filed Mar. 20, 2023 titled System and Method for Diagnostic Coding, which is related to U.S. patent Ser. No. 17/477,155 filed Sep. 16, 2021 titled System and Method for Diagnostic Coding, which claims the priority of provisional patent application U.S. Ser. No. 63/084,278 filed Sep. 28, 2020, titled “A checklist of elements for medical billing coding that audits what must be documented in the chart” and which is incorporated in its entirety herein by reference.

### BACKGROUND OF THE INVENTION

This invention relates generally to medical billing systems and, more particularly, to a medical diagnostic coding system and method that mandates correct claims coding of a medical diagnosis and provides instant feedback to and correction by a treating provider.

Medical claim and billing systems are inherently complicated, multi-layered, redundant, and inefficient. In a typical system, a treating provider makes a preliminary diagnosis which is then matched with a billing code, such as by a “coder.” The provider's diagnosis and code may then be reviewed by a clinic or hospital coding group and, if the code is found to be incorrectly selected, the “clinic or hospital coder” may change the code. The treating provider is typically never informed of the mistake and is likely to continue the incorrect coding practice. The clinic or hospital coder may then submit a claim to a respective insurance company where the diagnosis and code are once again reviewed—this time by an “insurance coder department which is composed of coders, nurses and physician.” If the code is still incorrect, the claim will be denied. The substitute diagnosis is given by the insurance company and the diagnosis/diagnoses are paid. Once the clinic or hospital are made aware of this, their claims department writes an appeal or dispute to substantiate the original diagnostic code(s). The clinic or hospital appeals department documents criteria that they believe will allow the original diagnostic code(s) to be reimbursed.

Various medical billing systems have been proposed throughout the years. Although presumably effective for their intended purposes, a common characteristic of such systems is that they still include multiple layers of claim audits, coding changes, and a lack of feedback to the treating provider.

Therefore, it would be desirable to have system and method that mandates correct diagnostic coding during a patient's first experience with a treating provider so as to minimize or even eliminate the multiple layers of claim audits, coding changes, and reimbursement modifications.

### SUMMARY OF THE INVENTION

A computer implemented coding system according to the present invention for mandating correct medical coding by a treating provider, comprises a processor for executing program code and a non-transitory computer-readable storage medium containing program code executable to perform the steps of receiving medical symptom data, examination findings, and medical diagnostic data associated with the patient so that the coding system can make a preliminary diagnosis of a medical condition of the patient. This enables a diagnosis to be determined based solely on the diagnostic elements performed during the patient encounter when matched with a matching record in a diagnostic code requirement database, said comparing and matching being performed in some embodiments using deep machine learning and artificial intelligence in real time for transforming the diagnostic guidelines via medical data promulgated by national authorities, learned articles, payor denial letter, or the like.

According to the present invention, the treating provider is asked to either accept the medical diagnosis based solely on the diagnostic elements that were generated by the initial patient encounter and which matches the guidelines found in the diagnostic code database or via operation of a machine learning/artificial intelligence engine diagnostic guidelines assimilated from UpToDate, Society Guidelines, and National Library of Medicine. If accepted, a code associated with the accepted diagnosis may be chosen and submitted to a payor with confidence of its correctness, and appropriate services ordered for the patient. If the diagnosis is not accepted, by contrast, the treating provider is invited to submit a physician suspected diagnosis (PSD) which is then matched to an appropriate diagnostic guideline in the manner described above and such that a list of “missing diagnostic elements” is determined and which are required by the PSD. Again, the treating provider is asked to either accept the originally determined diagnosis or else the missing diagnostic elements are identified and can be automatically ordered and the inventive method is essentially restarted.

Therefore, a general object of this invention is to provide a system for diagnostic medical coding that results in determining a diagnosis and associated billing code based solely on the initial patient encounter and without human interaction, said patient encounter including initial diagnostic elements that are matched with an up-to-date diagnostic guideline without needing a clinic, hospital or insurance audit procedure.

Another object of this invention is to provide a diagnostic medical coding system, as aforesaid, that matches initial diagnostic procedures and results with an up-to-date guideline and associated illness having identical diagnostic elements, thereby eliminating the risk of over-diagnosis, under-diagnosis or misdiagnosis.

Still another object of this invention is to provide a diagnostic medical coding system, as aforesaid, that seeks the approval or acceptance of the treating provider only after a diagnosis has been determined based solely on the diagnostic elements initially ordered and the results.

Yet another object of this invention is to provide a diagnostic medical coding system, as aforesaid, that provides immediate feedback to a treating provider regarding missing diagnostic elements that must be ordered or identified should the treating provider insists on making an independent diagnosis beyond that which is justified by the diagnostic elements performed initially.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1 is a block diagram illustrating a computer-implemented diagnostic coding system according to the preferred embodiment of the present invention connected to key player databases via the Internet;

FIG. 2 is a block diagram illustrating a coding database, a component of the diagnostic coding system as in FIG. 1;

FIG. 3 is a block diagram illustrating the diagnostic coding system according to the present invention in use installed on an electronic device having a processor and memory;

FIG. 4 is a flowchart illustrating a traditional diagnostic coding system;

FIGS. 5 to 7a is a flowchart of a process illustrating the diagnostic coding system according to the present invention;

FIG. 8 is a diagram of a supervised deep learning neural network according to the present invention;

FIG. 9 is another diagram of the deep learning neural network;

FIG. 10 is another diagram of the deep learning neural network; and

FIG. 11 is another diagram of the deep learning neural network.

### DESCRIPTION OF THE PREFERRED EMBODIMENT

A system and method for diagnostic coding according to a preferred embodiment of the present invention will now be described with reference to the accompanying drawings. The system 10 that mandates correct diagnostic coding includes computer software and a diagnostic coding database 20 connected to the internet and including coding executable by a processor 12.

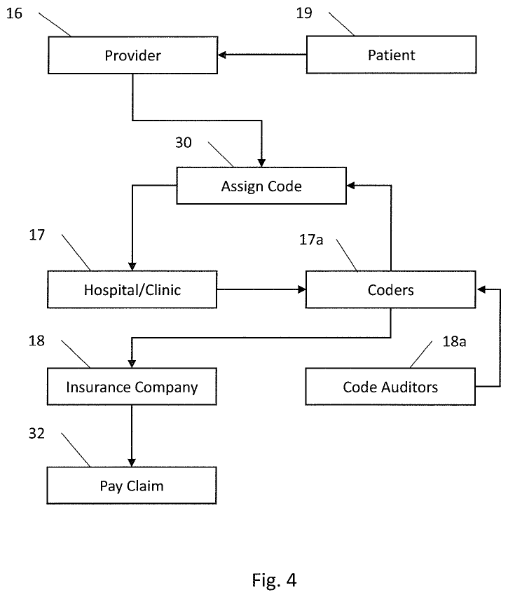

The system for diagnostic coding 10 may be stored on a non-transitory computer readable storage medium, i.e., memory 13, for execution by a processor 12 of an electronic device connectable to a wide area network 14 such as the Internet or local area network such as that of a clinic or hospital. Preferably, the diagnostic coding system 10 is stored in the cloud and accessed on an electronic device carried by a treating provider, such as a desktop computer, laptop computer, a tablet, or smart phone and that is connected to the network. The diagnostic coding system 10 may include program code 15 (e.g., computer instructions) and a database that includes diagnostic code requirement data 11 (FIG. 3). As will be discussed in greater detail later, the diagnostic code requirement data 11 may include a plurality of numeric diagnostic codes each being associated with a detailed outline of medical tests and medical procedures that must be administered or satisfied in order for a respective diagnostic code to be selected. In an embodiment, the diagnostic code requirement data 11, also known as a diagnostic element database, may receive repeated updates from authoritative sources such as the UpToDate, Society Guidelines, National Library of Medicine (NLM) and the National Institutes of Health (NIH), dataset libraries, SCOPUS, Web of science such that the diagnostic code database 11 is essentially a biomedical library that may be compared to respective diagnostic procedures and results of a patient encounter, whereby to determine a correct diagnosis of a patient's illness based solely on those initial diagnostic procedures and results, as will be described in greater detail later. As will also be described, this process of repeatedly updating the diagnostic element database 11 may include actual and specific algorithms that are “trained” using machine learning, artificial intelligence, and pattern recognition technologies in a machine learning engine environment.

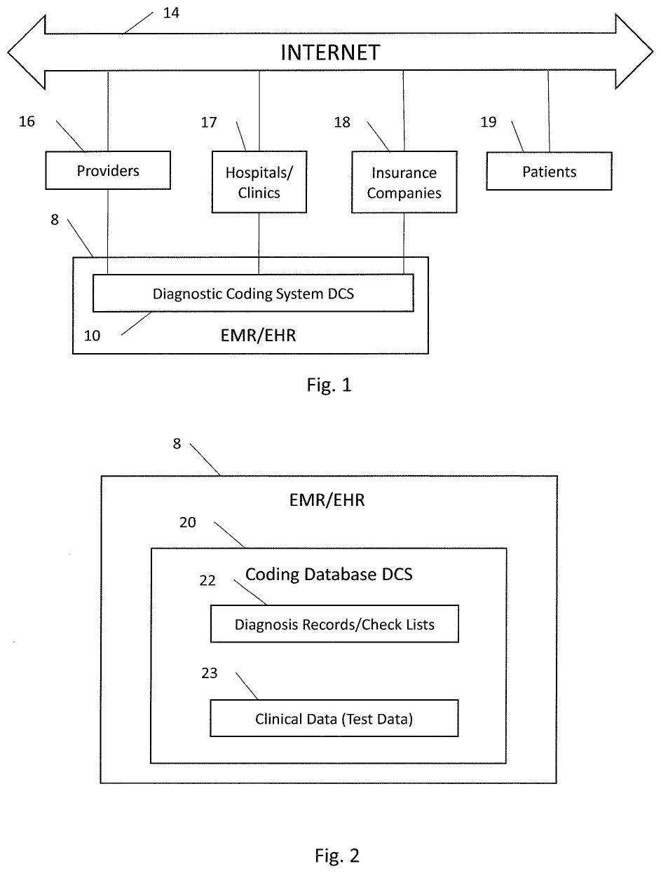

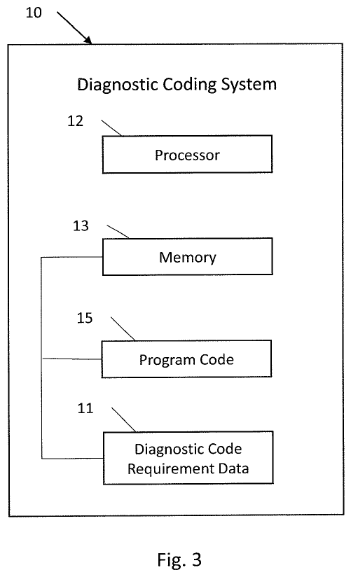

As shown particularly in FIGS. 1 and 2, it will be understood that the system for diagnostic coding 10 or at least the program code 15 will be resident in the cloud and may interface with the larger computer architecture of an Electronic Medical Record 8 (EMR), also known as an Electronic Health Record (EHR). An EMR is a digital version of the paper charts in the clinician's office. EMRs contain the medical and treatment history of the patients in one practice. As shown, the present system for diagnostic coding 10 may be a subset, program subroutine, or specialty program that plugs into the EMR 8.

Each installation and operation of the diagnostic coding system 10 may include a communications module in data communication with a wide area network such as the Internet 14 or with a local computer network such as within a clinic, hospital or hospital group. This preferred connectivity enables the diagnostic coding system 10 to be used by the treating provider 16, clinic, hospitals 17 or hospital coding groups 17a, insurance companies 18 or insurance company coding groups 18a, as well as directly with a consumer/patient 19 who may be in communication with the treating provider. In addition, this network connection enables digital communication with a source of diagnostic codes and elements and required diagnostic elements associated with each code, such as is provided by UpToDate, Society Guidelines, and the National Library of Medicine, National Institute of Health, dataset libraries, SCOPUS, and Web of science. Another network connection may be made with a payor rejection letter and its elements as will be explained later.

With specific reference to FIG. 2, the diagnostic coding system 10 includes a coding database 20 having a plurality of diagnosis records 22 each of which is associated with an alphanumeric code and with a comprehensive checklist or outline of symptoms, physical examination findings, medical tests and medical procedures that must be completed or satisfied in order for the respective code to be used to describe a respective patient's diagnosis. It is understood that the coding database 20 may be stored in a non-transitory storage medium, i.e., a memory device of the electronic device upon which the diagnostic coding system 10 is installed. Program code 15 may also be stored in the memory device 13 and configured for execution by a processor 12 so as to carry out the present invention. Further, actual clinical test data 23 (relative to the hypothetical patient who presents himself for treatment) may also be stored in appropriate data structures of the memory device. As will be explained later, the diagnostic records associated with a claim code or diagnostic code will be updated in association with using an artificial intelligence engine.

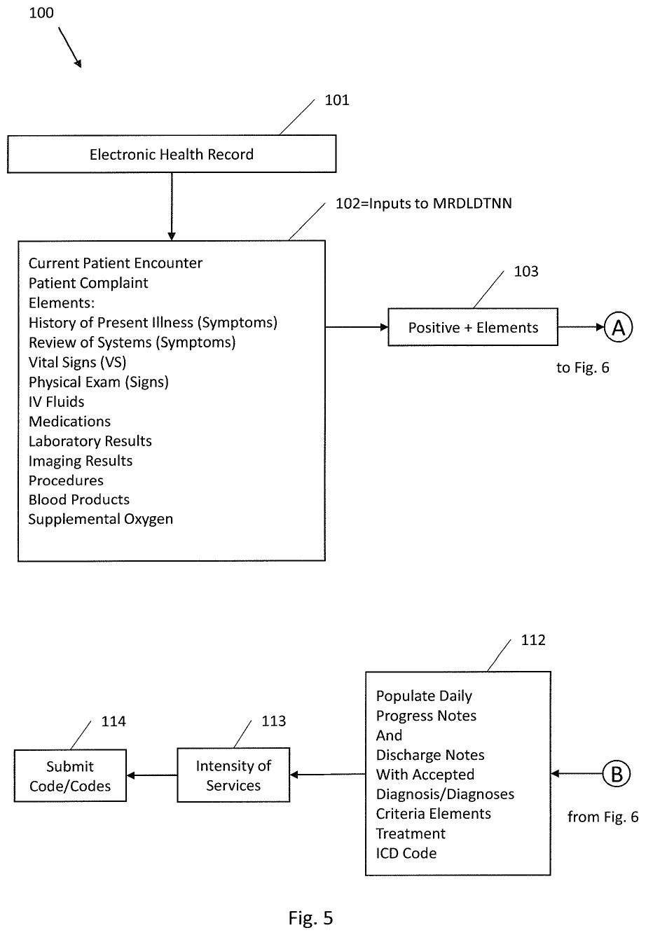

FIG. 4 illustrates the inefficiencies of a traditional diagnostic coding scheme and that which is typical of the prior art. With regard to a traditional diagnostic and billing system shown in FIG. 4, a treating provider 16 interacts with a medical patient 19, such as in person, via telemedicine, or the like. Based solely on an initial examination, a patient's report, or whatever preliminary tests may be run in real time, the treating provider 16 may make a preliminary diagnosis of a medical condition and may choose a diagnosis code accordingly. But before the selected diagnostic code can be submitted as an insurance claim, the provider's preliminary diagnosis may be submitted to a clinic/hospital 17 and, more particularly, to office or hospital coding group 17a where the preliminary diagnosis is considered along with any auxiliary medical test data that may have come into the patient's record after the preliminary diagnosis was made. The clinic or hospital coding group 17a may determine, based on a totality of the medical record, that the initial billing code is now incorrect. Similarly, the clinic or hospital coding group 17a may determine that the diagnosis is not clear and request or substitute another diagnosis code. In either instance, the clinic or hospital coders 17a may send the matter back to the treating provider 16 for reevaluation and recoding.

Once the clinic or hospital coding group 17a is satisfied with the diagnostic code, a claim may be submitted to the patient's insurance company 18 and, more particularly, to the insurance company coding auditors 18a. Once again, there is the potential that the insurance company coding auditors 18a may disagree that the claim diagnosis billed for is not supported by the documentation in the medical record. In such case, the insurance company coding department 18a will substitute another diagnosis and pay the hospital or clinic based on the substituted diagnosis.

As a real-world example of a patient encounter in a traditional billing system (FIG. 4), a patient may present to a provider regarding painful urination. The provider, then, may make an initial diagnosis of a “urinary tract infection” and the associated billing code is assigned. Unfortunately, however, this code may be premature until a plurality of additional testing has been completed and criteria elements have been identified. Currently, later lab data may reveal or even contradict this coding error which must then be changed after one or more coding audits by entities other than the treating provider, e.g., by clinic, hospital, or insurance company coding audits. In other words, a code assigned to “urinary tract infection” may require a different set of findings, lab tests, other tests, etc. that, if they had been performed, may have resulted in a different diagnosis and, therefore, a more appropriate coding. By contrast, this preliminary error is prevented by the present invention as discussed below.

By contrast, the efficiencies of the present invention will be summarized before being described in detail and then illustrated by way of the same example used with the inefficient traditional system described above. More particularly, a patient is thoroughly examined by a provider so as to hear and document the complaints of a patient, i.e., the initial patient encounter or patient complaint. This examination may prompt the provider to run an initial series of tests. In a critical aspect of the present invention, the software of the preferred embodiment of the diagnostic coding system 10, will correctly determine a preliminary diagnosis—independent of the treating provider. More particularly, the diagnostic coding system 10 is configured (such as by program code and using the diagnostic code requirement database 11) to generate a Medical Record Deep Learning Diagnosis, Diagnoses & Treatment Neural Network (MRDLDTNN). Stated another way, the present invention, i.e., the computer assisted coding system 10, is configured to determine the only diagnosis possible based on the preliminary tests and procedures shown to have been run at that point in time. In other words, the medical provider, e.g., the doctor, is simply not allowed to over diagnose or incorrectly diagnose the patient's malady and an incorrect diagnosis is not allowed to be submitted. Needless to say, the patient is not in danger of being incorrectly treated, such as with medicines or procedures. As will be described later, the determination of the MRDLDTNN may be made with use of a machine learning and artificial intelligence engine that is trained by repeated communications with diagnostic guidelines produced by an official source, such as UpToDate, Society Guidelines, National Library of Medicine, National Institute of Health, Dataset libraries, SCOPUS, Web of Science which provides diagnosis elements that must be undertaken before a code may be adopted, respectively. In fact, the machine learning/artificial intelligence engine may be trained by receiving repeated infusions or downloads regarding biomedical research, denial letters, and information concerning a correct diagnosis based on the diagnostic elements that have been performed. It is understood that the numerous diagnostic elements associated with a chosen diagnosis evolve and change over time, making the search engine all the more important to generate the correct preliminary diagnosis.

In another critical aspect, the computer-generated diagnosis is electronically presented to the medical provider and the medical provider is asked whether he accepts the computer-generated diagnosis (which will be referred to as the Medical Record Deep Learning Diagnosis, Diagnoses & Treatment Neural Network (MRDLDTNN)). Several post-patient-visit procedures take place and the proper code is submitted for payment. By contrast, if the MRDLDTNN is not accepted, then the ML/AI may be accessed again for the purpose of generating a list of diagnostic elements associated with the Physician Suspected diagnosis/diagnoses (PSD) which are displayed to the physician again, these additional elements are presented to the medical provider who is given the choice to accept the MRDLDTNN or to press on with his own diagnosis. If accepted, the process proceeds to the post-patient-visit procedures; otherwise, an order is generated for the missing diagnostic elements, i.e., an order for additional lab tests/procedures, etc., and the process essentially starts over.

This methodology has the advantages of providing knowledge to the treating provider regarding the complete outline of tests, procedures, etc., required for a given diagnosis and also enables the diagnosis to be changed based on the results of the required listing of tests. This process provides the instant feedback that a provider may need to avoid making a repeated error in diagnostic coding. Further, this process assures that the final billing code selected by the treating provider is correct-making future coding audits unnecessary. Accordingly, the final diagnostic code may be submitted to a clinic or hospital billing department then submitted as a claim to the insurance coding group for payment of the claim with total assurance that an audit is not necessary and that there is no need to return the coding issue back up the line.

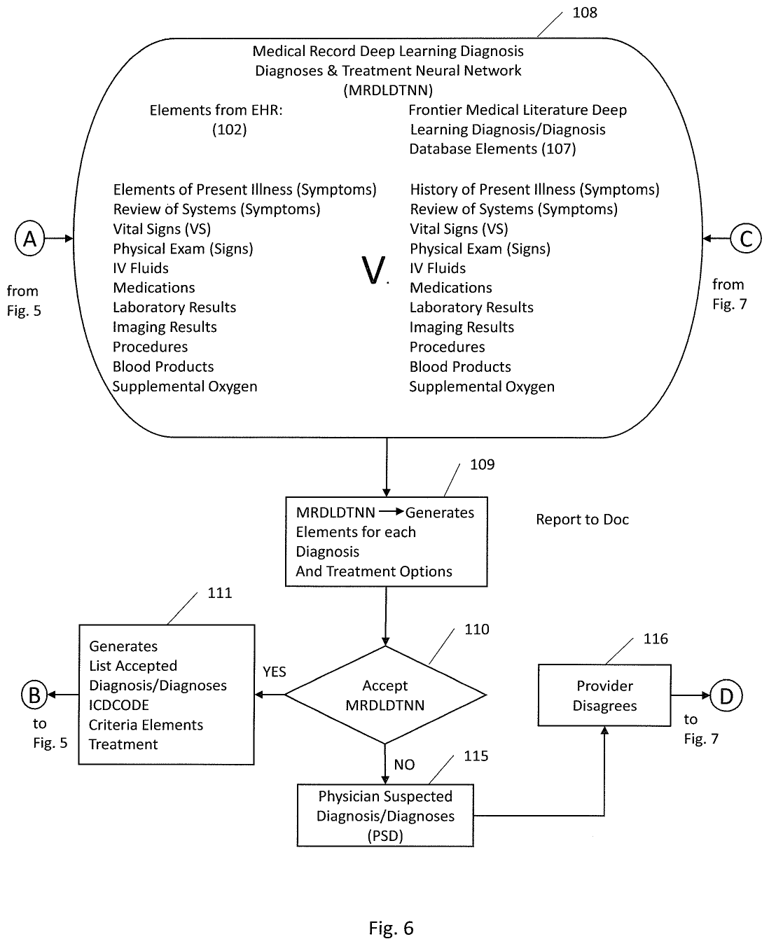

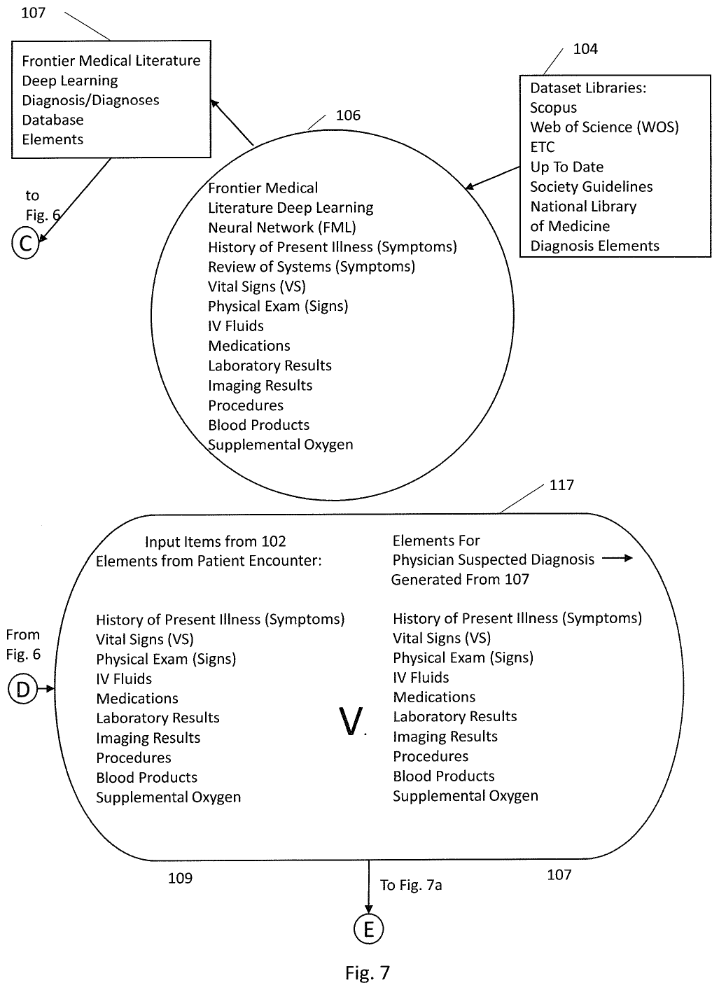

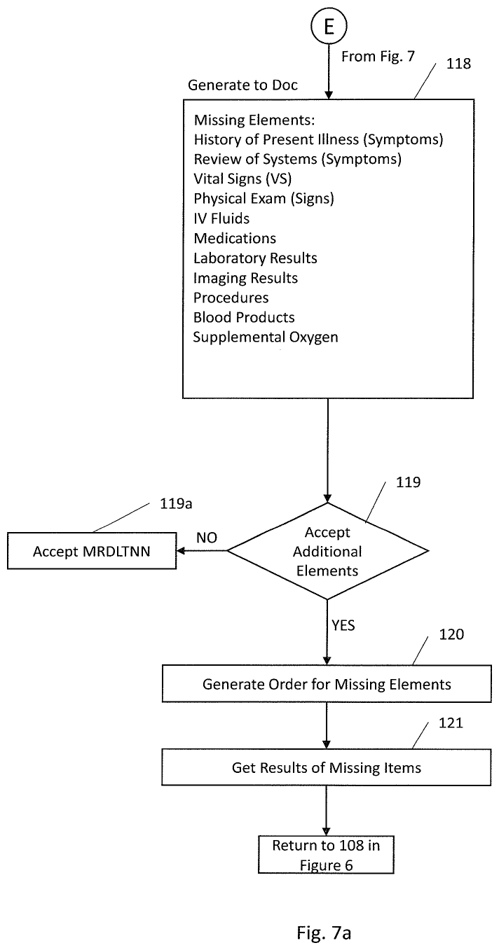

With the understanding explained above, this process and method will now be described in detail with reference to FIGS. 5 to 7a and with corresponding reference numerals.

A process 100 illustrating the logic of the diagnostic coding system 10 is shown in FIG. 5 et seq. It is understood that the process 100 is preferably implemented as computer software having a plurality of program instructions (aka programming) stored in a non-transitory memory 13 and executable by a processor 12 of an electronic device used or carried by a treating provider and that is connected to the Internet 14. According to the process 100 and as indicated at block 101, a patient may present himself to a treating provider and presumably brings his personal electronic health record (EMR) with him (which may be in the form of electronic records in the computer system of the provider, on a flash drive belonging to the patient, or otherwise electronically archived) so that it is available to the treating provider and may, if appropriate, be supplemented during or after the client encounter. As shown at block 102, a patient encounter occurs when a patient presents his symptoms to the treating provider, such as in a typical office visit, or hospital setting, a telemedicine encounter over the Internet 14, or the like. It is understood that a complete patient encounter, which may also be referred to as a “patient complaint,” may include recording a history of the present illness (i.e., a patient discussing his symptoms), a review of a patient's bodily systems, the taking of the patient's vital signs, and a physical examination, a review of the patient's IV fluids (or an order is made directing that IV fluids be given), a review of the patient's current medications, the review of any imaging results (such as x-ray, CT scan, MRI scan, or the like), laboratory results, a review of any medical procedures undertaken, a review of any blood products needed and given, and any supplemental oxygen. The process 100 proceeds to step 103.

At step 103, the exact nature and results of the diagnostic elements currently in the patient's encounter are determined—no more and no less. The diagnostic elements by which the initial diagnosis may be determined have been completed by way of the initial patient encounter. The process 100 proceeds to step 108 via 103.

At step 108, the processor 12 determines what diagnosis is indicated by the diagnostic elements resulting from the patient encounter determined in step 103. Control is passed to step 108. Naturally, step 108 involves comparing the diagnostic elements (and their results) with the predetermined diagnostic elements prescribed by a standardized medical authority, such as Frontier Medical Literature Deep Learning Diagnosis/Diagnoses Database (FML). Note that the comparison of diagnostic elements from the patient encounter versus a Medical Record Deep Learning Diagnosis/Diagnoses & Treatment Neural Network (MRDLDTNN) is shown in block 108. Block 108 is intentionally sandwiched between block 103 and block 107 in that the comparison of these two sets of elements occurs in block 108. And, the MRDLDTNN diagnostic elements may be generated by the actions shown in blocks 104 and 106. More particularly, block 106 represents a Frontier Medical Literature Deep Learning Neural Network that is uniquely configured to generate a more complete and accurate set of guidelines to be used in step 107 and then step 108. More particularly, the FML engine is trained by its repeated network access to the latest diagnostic elements, biomedical research, biomedical articles, and the like proffered by UpToDate, Society Guidelines National Library of Medicine (step 104). The action of training a neural network with hidden layers and data sets will be described in greater detail later. In addition, and as shown at step 106, the deep learning neural network may receive and be trained by the latest expert information regarding the history of the present illness indicated by the patient's symptoms and any elements determined so far.

Further, when the processor 12 compares the computer-generated diagnostic elements to the patient encounter diagnostic elements in step 108, the processor 12 will determine at step 109 what is referred to as a MRDLDTNN (ML/AI Diagnoses/diagnoses), also known as the computer-generated preliminary diagnosis based solely on the patient encounter synthesized in comparison with the diagnostic elements that have been implicated so far—nothing more and nothing less. Preferably, each element of the MRDLDTNN will be graphically or textually matched with each corresponding diagnostic element that was performed during the patient encounter so as to more powerfully justify the correctness of the preliminary diagnosis (a.k.a. the MRDLDTNN diagnosis was correct). Such a comparison may utilize pattern recognition and other algorithms that are characteristic of the FML. The process 100 proceeds to step 110.

At step 110, the processor 12 determines if the treating provider will agree to accept the MRDLDTNN discussed above (such as by posing this question in a text will prompt on-screen) and, if so, the process 100 proceeds to steps 111, 112, 113 and 114 at which the accepted diagnosis may be saved or published, post-encounter services may be prompted/ordered, and the accepted diagnostic/insurance code may be submitted for payment, respectively. (FIGS. 5-7a). But, if the MRDLDTNN is not accepted by the treating provider at step 110, the process 100 proceeds to step 115 and 116.

At step 115, the treating provider may input his suspected diagnosis (also referred to as a Physician Suspected Diagnosis (PSD)) in the process 100 proceeds to step 117. At step 117, the ML/AI/PR engine is configured to synthesize or make a comparison of the diagnostic elements generated by the patient encounter (as described previously) with the diagnostic elements associated with the PSD so as to generate a set that will be referred to and saved as “Missing Elements” at step 118. In other words, the processor 12 is configured to determine, using the AI engine (which, again, may be in the data communication with UpToDate, society guidelines, SCOPUS, Web of Science and the National Library of Medicine) a list of diagnostic elements (i.e., test procedures, etc.) that are uniquely required for the PSD but that have not been accomplished per the patient encounter and MRDLDTNN. After saving and publishing this list of Missing Elements at step 118, the process 100 proceeds to step 119.

At step 119, the processor 12, in a routine similar to that described above in step 110, queries the treating provider to determine if the treating provider agrees to proceed in ordering the Missing Elements (i.e., agrees to order the missing lab tests or medical procedures, etc. that are prescribed for the PSD) and, if not, the process 100 proceeds to step 119a and the MRDLDTNN (original CGDDDE) is accepted/adopted. But, if the treating provider agrees that the Missing Elements that need to be considered before the PSD can be confirmed, then the process proceeds to step 120 whereby orders for the additional elements are generated in the process proceeds to step 121 where the procedures according to the Missing Elements take place in what will be referred to as a secondary or auxiliary or supplemental patient encounter. Accordingly, the data from the auxiliary or supplemental patient encounter may update the EMR and the process returns to step 108 where a new MRDLDTNN may be determined at step 109 and presented for acceptance at step 110 as described above.

In an embodiment, the medical guidelines relied upon for determining a correct diagnosis and assignment of a corresponding diagnostic code may be refined, updated, transformed, and improved in real time through incorporation of a deep learning model of artificial intelligence. To be thorough in its disclosure, the concept of machine learning and artificial intelligence will be described in greater detail. In the last decade, the applications of machine learning (ML) algorithms have proliferated in many areas of scientific, industrial, and medical applications. In general, modern artificial intelligence (AI) systems are capable to classify with high precision very complex data. The algorithms have inspired the development of a novel artificial neural network (ANN or NN) forming the basis of the field of artificial intelligence (AI). The developments have been in response to the vast amount of data, known as Big Data, which require new approaches to their processing, storage, and information extraction. Not without some limitations, AI continues to find new applications and provide fast information extraction based on algorithm embedded knowledge acquired with the use of available data in order to process newly available data: the process also known as NN training.

Machine learning (ML) is a dynamic field of artificial intelligence (AI) that empower systems to learn from data and improve their performance over time without explicit programming. The four fundamental types of machine learning are supervised learning, unsupervised learning, semi-supervised learning, and reinforcement learning. Many of these terms will be defined here and used appropriately in later discussions as the present invention presents a deep learning model of artificial intelligence that incorporates many of these concepts. For instance, supervised learning involves training a model on a labeled data set, where each input is associated with a corresponding output. During training, the model learns to map inputs into outputs by adjusting its parameters. The objective is to generalize to new, unseen data. Supervised learning is used for applications such as classification, speech recognition, and regression analysis. Further, unsupervised learning operates on unlabeled data with the intent to discover inherent patterns, structures, or relationships within a data set. Complex algorithms may identify similarities, groupings, or anomalies without explicit guidance. It should be appreciated that both of the above-described forms of machine learning are more effective at identifying subtleties, nuances, and complex medical diagnoses with accuracy never before possible by human intellect alone.

Further, semi-supervised learning combines elements of both supervised and unsupervised learning, utilizing a data set with a small portion of labeled examples alongside a larger pool of unlabeled data. This type of learning is advantageous in medical image analysis. Finally, reinforcement learning involves training and agent to make sequential decisions in an environment so as to maximize a cumulative reward signal. It will be appreciated that various forms of machine learning each have their strengths and weaknesses and, when used together, can generate powerful and lifesaving results in improved patient care.

In another important aspect, the present deep learning model may utilize federated machine learning [symbol] an approach that enables training models across decentralized devices or servers holding local data samples. In other words, all of the data that is gathered in the present model is not necessarily gathered from one place but FML allow the model to be trained across multiple electronic devices that each holds local data sets. In the present invention, for instance, medical data may be gathered from medical articles, research papers, seminar summaries, medical libraries, insurance coding databases, and the like so as to generate data sets applicable to patient medical data, i.e. from tests, lab work, and patient examination preferably, the process of gathering large amounts of data from a myriad of sources occurs in real time and continuously as this body of knowledge is ever evolving. It will be understood that all of the above referenced sources of medical data may be included when the legal claims recited below recite “at least medical articles.”

In another important aspect, medical data may be received and interpreted using natural language processing paradigms. More particularly, the preliminary and auxiliary medical test data may be scanned and received into a natural language (NL) reader. In general, natural language processing (NLP) refers to using machine learning algorithms in relation to text and speech. More particularly, NLP may be used to create machine translations, document summaries, question answering, predictive typing, and the like. In the present case, medical documents summarizing lab work, medical advice, and other documentation may be scanned in or digitally transferred to a natural language file to be viewed by the treating provider or compared to the diagnostic code database 20 whereby the processor 12, executing programming code 15, is able to determine if a selected diagnostic code associated with a respective record and associated diagnosis corresponds with scanned medical test data and diagnosis (initial or final).

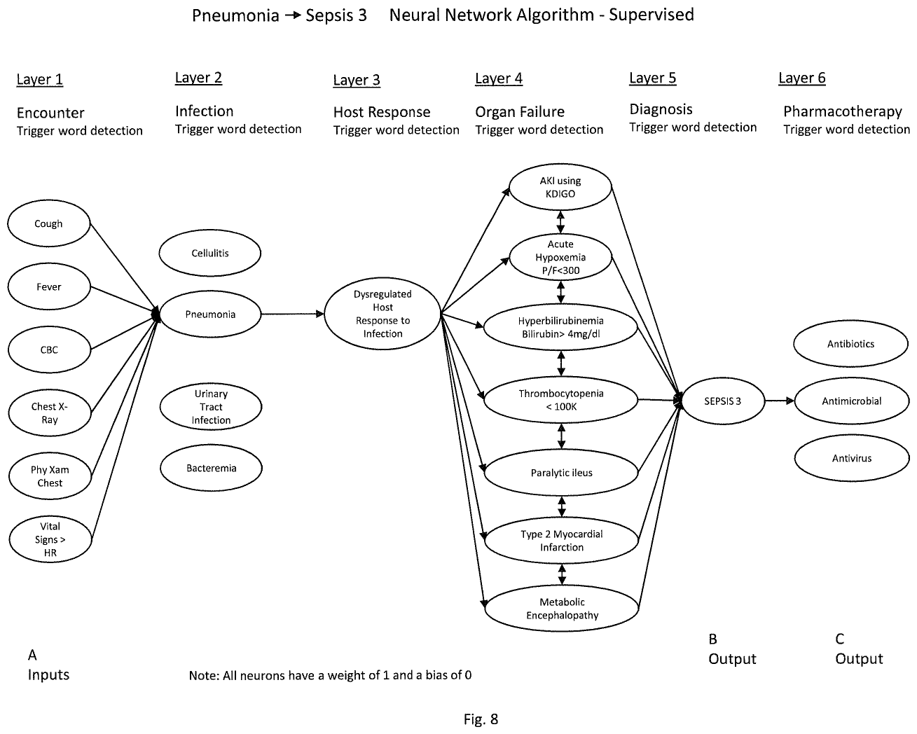

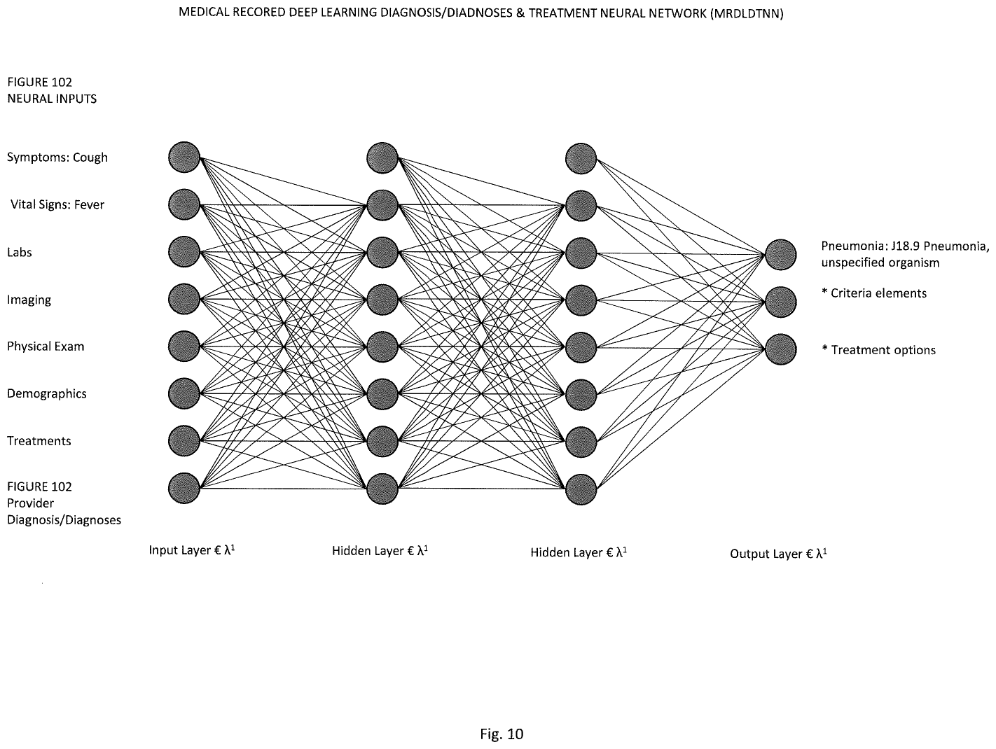

With this background of descriptions and definitions and with specific reference to FIGS. 8 through 11, the preferred deep learning model of the present invention will be described in greater detail. This patent utilizes Deep Learning, (a subfield of Machine Learning, which is a subfield of Artificial Intelligence) for enhanced and optimized accuracy and consistency of the medical diagnosis and resulting International Classification of Diseases coding (ICD), resulting in more accurate medical records, treatments, more accurate health care risk score analysis forecasting, billing, health informatics, and statistics. Deep learning is needed to process the huge amount of structured data that is gathered. Again, structured data is data that has a standardized format for efficient access by software and humans alike. In healthcare, structured data may include demographic information (first and last name, date of birth, home address, gender), vital signs (height, weight, blood pressure, blood glucose), and data elements such as diagnostic or billing codes, medications, and laboratory test result. Further, the present model gathers unstructured medical data. Unstructured data includes images, documents, audio, video, and text that is analyzed using tools such as NLP (natural language processing) to extract information which can be used by the algorithms [symbol] in real time. Preferably, the present model is running 24/7/365 (i.e., real-time) to analyze the structured and unstructured data to provide the most accurate diagnostic criteria) which is required for this invention. Complex algorithms (which include Convolutional Neural Networks (CNNs), Long Short Term Memory Networks (LSTMs), Recurrent Neural Networks (RNNs), Generative Adversarial Networks (GANs), Radial Basis Function Networks (RBFNs), Multilayer Perceptrons (MLPs), Self-Organizing Maps (SOMs) can be used for clustering data without knowing the class memberships of the input data, Deep Belief Networks (DBNs) will train the model (i.e., teach the model to correctly interpret data and learn from it). In order to perform a task accurately, the Deep Learning model may extract features through embedding language libraries from the medical literature to analyze the input to extract features and identify similar objects. This model can perform tasks that normally require human intelligence, such as decision making, object detection, and solving complex problems concurrently while evaluating a patient during each patient encounter. However, due to the epochs that Deep Learning completes (the number of epochs is a hyperparameter that defines the number of times that the learning algorithm will work through in the entire training dataset); the number of epochs is traditionally large allowing the learning algorithm to run until the error from the model has been sufficiently minimized.), its accuracy exceeds that of any human neural network through artificial general intelligence. This process is performed concurrently as the medical record is being completed. The existing system is inferior to this process because of human medical provider error, i.e., providers are unable to “see” the missing elements or patterns required to make accurate diagnostic assessments resulting in over-diagnosis and under-diagnosis in the current system, i.e., misdiagnosis.

The deep learning model neural networks will provide the analysis, calculations, and output using a hybrid model composed of a convolutional neural network, (CNN). A convolutional neural network (CNN or ConvNet) is a network architecture for deep learning that learns directly from data. CNNs are particularly useful for finding patterns in images to recognize objects, classes, and categories. CNN can be quite effective for classifying audio, time-series, and signal data. The deep learning model may also use a recurrent neural network, (RNN). A recurrent neural network is a type of artificial neural network which uses sequential data or time series data. RNNs are designed to handle input sequences of variable length, which makes them well-suited for tasks such as speech recognition, natural language processing, and time series analysis). Further, the deep learning model may use multilayer perceptrons (MLP). Multilayer Perceptron is a Neural Network that learns the relationship between linear and non-linear data, generative models. Generative AI is a type of artificial intelligence technology that can produce various types of content, including text, imagery, audio and synthetic data. Further, the deep learning model may utilize deep belief networks. A deep belief network (DBN) is a probabilistic, generative model made up of multiple layers of hidden units to provide the deep learning-based disease diagnosis with ICD code with optimal accuracy.

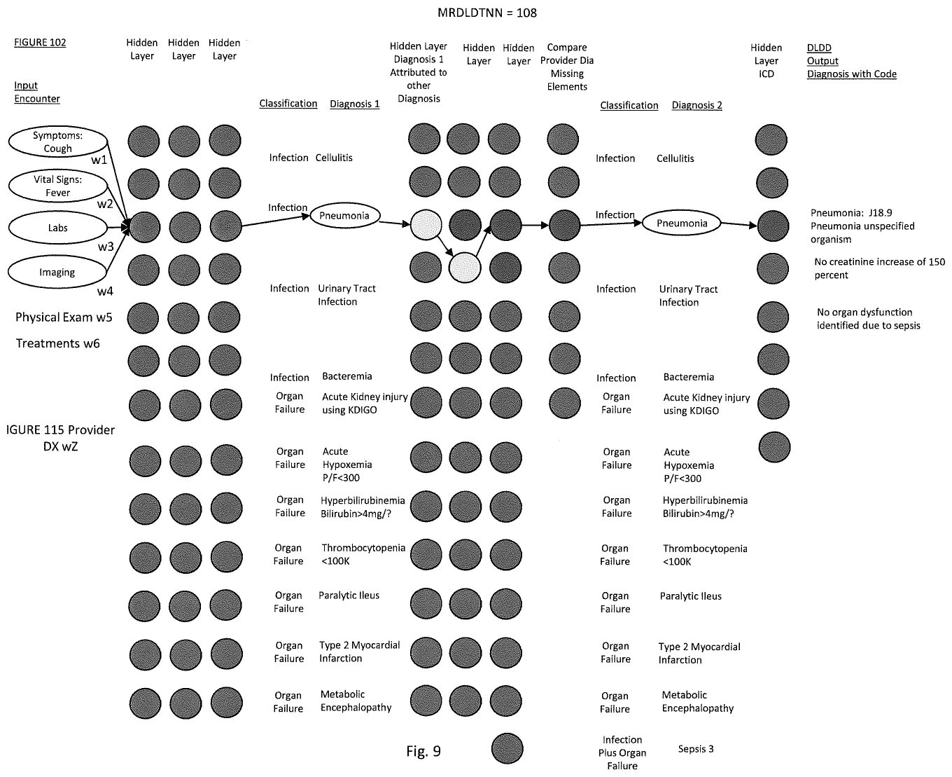

Due to the complexity of the data, each neural network is needed for analysis, calculations, and predictions. The initial training data will be a portion of the total training data, i.e., a collection of labeled information that will be used to build the machine learning model. This labelled data used in the supervised learning stage will include annotated text, images, numerical values, categorical values, and video (See FIG. 8). Labeled data is raw data that has been assigned labels to add context or meaning, which is used to train machine learning models in supervised learning. This training data will interact with multiple layers of calculations in hidden layers to extract information from the input. Hidden layers refer to the intermediary stages between input and output in a neural network. They are responsible for learning the intricate structures in data and making neural networks a powerful tool for a wide range of applications, from image and speech recognition to natural language processing and beyond. Hidden layers in a neural network department are represented in FIGS. 8 and 9.

The input brings the initial data into the system for further processing by subsequent layers of artificial neurons which will interact with the recurrent neural network, RNN, natural language processing, sentiment analysis, DNA sequence classification, speech recognition, and language translation. The RNN recurrent neural network is a type of artificial neural network which uses sequential data or time series data. RNNs are designed to handle input sequences of variable length, which makes them well-suited for tasks such as speech recognition, natural language processing, and time series analysis), will utilize Long Short Term Networks (a deep learning, sequential neural network that allows information to persist) to allow information to persist for large datasets. The input will also interact with convolutional neural networks, A convolutional neural network (CNN or ConvNet) is a network architecture for deep learning that learns directly from data. CNNs are particularly useful for finding patterns in images to recognize objects, classes, and categories. CNN can be quite effective for classifying audio, time-series, and signal data) (CNN) for images. The supervised learning will also utilize support vector machines, (SVMs). SVMs are supervised learning models with associated learning algorithms that analyze data for classification and regression analysis for classification, regression, and outlier detection which can categorize unlabeled data. With this training data, the Deep Learning model will learn to perform its tasks at a high level of accuracy to develop predictive capability. This training data will interact with datasets imported from dataset libraries to include but not limited to Scopus, Web of Science (WOS), Google Scholar, Google DataSets, PubMed, Cleveland database, chronic kidney disease dataset, Pima diabetic dataset, Parkinsons dataset, WDBC dataset, Covid-chest x-ray dataset, and UCI machine learning repository. These datasets will be used multiple times, as epochs, throughout the training process to refine the model's predictions and improve its success rate through forward and backward propagation with activation functions to include but not limited to Sigmoid, ReLU, and modified ReLU. This will allow the output loss to be minimized through optimizer programs and gradient descent to manage the Learning Rate. This will be managed by programs including but not limited to ADAM (which stands for Adaptive Moment Estimation, is an adaptive learning rate algorithm designed to improve training speeds in deep neural networks and reach convergence quickly). Transfer learning (TL) is a technique that utilizes a trained model's knowledge to learn another set of data. Transfer learning aims to improve learning in the target domain by leveraging knowledge from the source domain and learning task. For example, learning to recognize a pulmonary embolism when the model has already been trained to recognize pneumonia. TL will be used to take knowledge learned from the supervised learning step to boost performance on the validation and test process. After the training process is completed, a small percentage of the original training data will be used to validate the model to determine whether it correctly identifies new data. At this stage, the hyperparameters will be adjusted to optimize the outputs. Lastly, the final data is fed to the model to confirm optimized output. The validation data, which is a small percentage of the original training data, will be used to determine how the model performs on new data to ensure that it performs accurately. In addition, Generative adversarial networks create new data instances that resemble your training data. A GAN based approach will be used to generate synthetic data which looks similar to real data if there is data scarcity to ensure optimization of the outputs.

For example, a patient presents to the emergency department for evaluation.

Input Layer variables for #102 include but are not limited to:
Age: 72
Ethnicity: African American
Sex: Male
Symptoms: fever, cough
Exam findings: Rales in lungs, rhonchi
Vital signs: temperature 38 C, heart rate 110, respiratory rate 20, oxygen saturation 85 percent on room air
Chest Imaging: infiltrates in right middle lung
Labs: WBC 18,000, creatinine 1.1
Treatment: Rocephin IV, azithromycin IV
Drug or other Allergies: Penicillin
Genetics: None identified

With respect to reference 103, positive elements are fed to the hidden layers. Negative input elements fall out and become Nonessential Elements. This is structured and unstructured data from the medical record. Further, reference 108 refers to hidden layers. Still further, reference number 109 references output from the Medical Record Deep Learning Diagnosis/Diagnoses & Treatment Neural Network (MRDLDTNN). Reference 106 refers to Frontier Medical Literature Deep Learning Neural Network Database (FML)

### Overview

Using a hybrid model composed of a convolutional neural network (CNN), recurrent neural network (RNN), multilayer perceptrons (MLP), generative models, and deep belief networks to provide the deep learning-based disease diagnosis with ICD code, criteria elements, and recommended literature-based treatment options with optimal accuracy for the 72,000 current ICD codes. Due to the complexity of the data, neural networks are needed for analysis, calculations, and predictions. This model will be resident in the “cloud” in a platform such as Microsoft Azure Cloud.

### Process 1: Data Gathering

Identification of the initial input neurons based on medical record information. The input characteristics include but are not limited to symptoms, vital signs, laboratory results, imaging, physical exam findings, demographics, treatments, and provider diagnosis (diagnoses). Embedding programs, to include, work, document, graph, image, and text embedding, and vector databases will identify text and semantics to convert the input neuron for fast retrieval and similarity search. Natural Language Processing, NLP, will also be used to convert the data to usable deep learning data. The input data is converted to information that can be analyzed by the computing program through these embedding programs which are vector databases to convert this information to data that can be manipulated in the hidden layers.

### Process 2: Preprocessing the Data

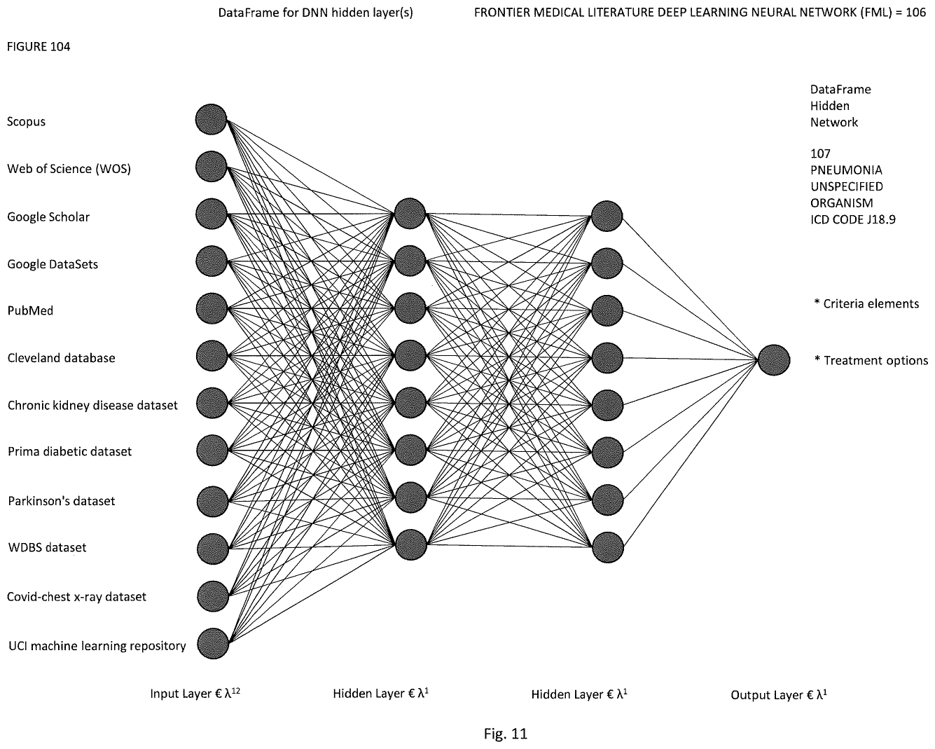

Step 1: Feed the input neurons to the network through channels which are represented as connecting lines on the diagram shown in FIG. 11. The initial parameters, inputs, are manually labelled and weighted as w₁, w₂, w_{Z}. This is needed to manually train the model using training data. The structured input neuron is converted to language that the neural network through embedding and vector databases that can process the input. Natural Language Processing will also be utilized when applicable. This is the beginning of the forward propagation process.
Step 2: Calculate the weighted sum of the inputs plus bias.
Step 3: The information from Step 2 is transferred to Step 3 through channels where the Activation function takes the weighted sum of input plus the bias as the input to the function and decides whether the neuron should be fired or not fired. This activation is based on one of several functions such the ReLU function, modified ReLU function and sigmoid function. The model is a threshold-based activation function. If Y value is greater than a certain value, the function is activated and fired or else not. This is an instantaneous function.

### Process 3: Training the Model

The model is trained with structured data in many epochs (The number of epochs is a hyperparameter that defines the number of times that the learning algorithm will work through the entire training dataset. The number of epochs is traditionally large allowing the learning algorithm to run until the error from the model has been sufficiently minimized), which is a complete iteration through the entire training dataset in one cycle for training the deep learning model, as are needed to ensure accuracy of the program through forward and backward propagation. This is continued until the accuracy is optimized for the training data.

### Process 4: Evaluation

Step 1: The output layer provides the Deep Learning generated diagnosis using the FML neural network database in the hidden layers, and the output is compared to the expected diagnosis. The loss is calculated using a loss function.
Step 2: Back propagation is performed to propagate the loss back through the network until the loss function is optimized. Initially this process will be Supervised until the Loss is optimized using an optimization model such as ADAM and using a recurrent neural network.
Step 3: The model is then tested using validation data and once this is optimized, then it is tested on testing data. The data will have cross validation to avoid overfitting.

Step 4: The output layer compares the chart diagnosis, an input, with the Deep Learning generated diagnosis and provides the input characteristics that are missing based on the hidden layers.

### Outputs:

In the neural network drawings (FIGS. 8 and 9), Diagnoses 1: generated by the clinician is pneumonia, sepsis, and acute kidney injury.

Diagnosis 2: Based on the example above, the valid diagnosis generated by Deep Learning Neural Network using the trained FML dataset includes only pneumonia, the ICD code, its criteria, and its treatment options. The missing elements of the provider diagnosis are generated. The provider can accept these missing elements and accepting this will generate an order in the medical record if the criteria elements must be ordered. The provider can also decline to accept the Deep Learning generated diagnosis.

Step 7: Once the diagnosis is accepted, the diagnosis input uses the hidden layer to generate the diagnosis, the ICD code, the criteria, and the recommended treatment options.

In use, the system and method for correct medical diagnostic coding 10 is configured to determine a correct initial medical diagnosis that is based solely on an initial patient encounter and the initial diagnostic elements that were performed during that initial patient encounter. More particularly, the treating provider is not given opportunity to over-diagnose, under-diagnose, or mis-diagnose; rather, the processor 12 in conjunction with the program code 15, diagnostic code requirement database 11 and, in some embodiments, the ML/AI/PR engine are configured to determine the proper initial diagnosis of the patient's malady according only to the diagnostic elements having already been carried out. In fact, this preliminary diagnosis, based only on the data of record i.e., the MRDLDTNN, will be published and presented to the treating provider along with an inquiry to determine if the provider agrees with the MRDLDTNN. If accepted, a few post-procedure actions will occur and the inventive method is terminated. Otherwise, the provider may enter his own suspected diagnosis, to which the processor 12 is configured to determine a list of diagnostic elements required for said provider diagnosis and to publish or display this list of Missing Elements to the physician along with an inquiry if the treating provider will either accept the original MRDLDTNN or, rather, agrees to order the additional or auxiliary diagnostic elements (e.g., Additional lab tests, scans, or other medical procedures). If the additional diagnostic elements are ordered, the results will be added to the patient encounter and the method of determining a diagnosis starts over. Importantly, the scenario of an initial diagnosis being determined by the system software and the results being presented to the treating provider has the unique advantage of providing immediate educational feedback to the treating provider and displaying the only possible diagnosis based on current medical elements currently of record may lead to supplemental and additional diagnostic elements to be ordered and considered before agreeing to a diagnosis and, therefore, an insurance code submitted for payment of a claim.

According to the present invention, a patient diagnosis can be made quickly and accurately without undertaking one or multiple coding audits.

It is understood that while certain forms of this invention have been illustrated and described, it is not limited thereto except insofar as such limitations are included in the following claims and allowable functional equivalents thereof.

1. A computer implemented system for determining a medical coding corresponding to a medical diagnostic code using at least one deep learning algorithm of artificial intelligence, said system comprising:
a non-transitory computer-readable storage medium containing program code and data structures;
a diagnostic coding database in said storage medium having a plurality of records each including at least a medical diagnosis and a corresponding outline of predetermined diagnostic elements that must be ordered and determined before a code associated with said medical diagnosis can be assigned;
a deep learning neural network in communication with said diagnostic coding database that is trained using medical data that is updated in real-time using at least medical literature obtained using natural language processing (NLP);
a processor in data communication with said computer-readable storage medium and operative to execute said program code to perform the steps of:
receiving medical symptom data from the patient;
receiving diagnostic test data associated with the patient;
comparing said received medical symptom data and said received diagnostic test data to said plurality of records of said diagnostic coding database and to said medical data obtained from said deep learning neural network until a mutually exclusively matching record is located;
publishing said matching record and a corresponding medical diagnosis and corresponding outline of predetermined diagnostic elements associated therewith;
determining if a treating provider agrees that the medical diagnosis associated with said matching record should be made final and, if so, recommending predetermined patient support information and, if not, receiving physician suspected diagnosis (PSD) data;
determining from said diagnostic coding database a respective record corresponding to said PSD data, said respective record including a PSD outline of diagnostic elements;
determining a list of missing diagnostic elements yet to be ordered or performed by comparing said PSD outline of diagnostic elements with said corresponding outline associated with said matching record; and
determining if the treating provider agrees that the medical diagnosis associated with said matching record should be made final and, if so, recommending predetermined patient support information and, if not, generating orders to perform said list of missing diagnostic elements.

2. The system as in claim 1, wherein said deep learning neural network is in electronic communication with said diagnostic coding database and said processor is configured to transform in real-time said plurality of records in said diagnostic coding database into a revised plurality of records and in accordance with the medical data received from said deep learning neural network.

3. The system as in claim 2, wherein said deep learning neural network includes supervised learning that operates on labeled data so as to map a plurality of inputs to a plurality of outputs each time adjusting corresponding parameters so as to generate modified data.

4. The system as in claim 3, wherein said deep learning neural network includes unsupervised learning that operates on unlabeled data so as to discover inherent patterns, structures, and relationships within a data set.

5. The system as in claim 4, wherein said deep learning neural network is trained using forward and backward propagation through a predetermined set of hidden layers and for a plurality of epochs/iterations corresponding to the number of hidden layers.

6. The system as in claim 5, wherein said deep learning neural network is trained using data obtained in real-time from a group that includes Google Database and corresponding Data Sets, Google Scholar, Google AI Library Database, UCI machine learning repository, Disease Diagnosis Dataset libraries, Scopus, PubMed, Cleveland database, chronic kidney disease dataset, Pima diabetic dataset, Parkinsons dataset, Breast Cancer Wisconsin (Diagnostic) Data Set (WDBC dataset), Covid-chest x-ray dataset, and Web of Science (WoS).

7. The system as in claim 1, wherein said deep learning neural network is a hybrid model that includes a convolutional neural network (CNN), a recurrent neural network (RNN), multilayer perceptron, deep belief networks (DBN), and generative AI.

8. The system as in claim 1, wherein said processor is configured to submit said received medical symptom data, said received diagnostic test data, said corresponding medical diagnosis and treatment options therefor as hidden layers of the deep learning neural network so as to generate a data set for submission to the Frontier Medical Literature Database framework so as to transform and improve medical data stored therein.

9. The system as in claim 1, wherein said processor is configured to execute said program code to perform the steps of:
using the diagnostic coding database to determine an insurance code associated with said accepted diagnosis associated with said matching record if the treating provider accepts that the medical diagnosis associated with said matching record should be made final; and displaying on a display screen said determined insurance code associated with said accepted diagnosis.

10. The system as in claim 9, wherein:
said processor is configured to execute said program code to perform the step of communicating said determined insurance code associated with said accepted diagnosis to a third-party coder via a wide area network;
said third party coder is an insurance company; and
said third party coder is a clinic or hospital.

11. A method for determining a medical coding corresponding to a medical diagnostic code using at least one deep learning algorithm of artificial intelligence, said method comprising:
receiving into a diagnostic database up-to-date diagnostic guidelines, each diagnostic guideline including at least a medical diagnosis and the corresponding outline of predetermined diagnostic actions that must be ordered and determined before a code associated with a medical diagnosis can be assigned;
providing a deep learning neural network in communication with said diagnostic coding database that is trained using medical data that is obtained and updated in real-time using medical literature obtained via a computer network using natural language processing (NLP);
receiving medical symptom data from the patient;
receiving diagnostic test data associated with the patient;
comparing said received medical symptom data and said received diagnostic test data to said plurality of records of said diagnostic coding database and to said medical data obtained from said deep learning neural network until a mutually exclusively matching record is located;
publishing said matching record and a corresponding medical diagnosis and corresponding outline of predetermined diagnostic elements associated therewith;
determining if a treating provider agrees that the medical diagnosis associated with said matching record should be made final and, if so, recommending predetermined patient support information and, if not, receiving physician suspected diagnosis (PSD) data;
determining from said diagnostic coding database a respective record corresponding to said PSD data, said respective record including a PSD outline of diagnostic elements;
determining a list of missing diagnostic elements yet to be ordered or performed by comparing said PSD outline of diagnostic elements with said corresponding outline associated with said matching record; and
determining if the treating provider agrees that the medical diagnosis associated with said matching record should be made final and, if so, recommending predetermined patient support information and, if not, generating orders to perform said list of missing diagnostic elements.

12. The method as in claim 11, wherein said deep learning neural network is in electronic communication with said diagnostic coding database so as to transform in real-time said plurality of records in said diagnostic coding database into a revised plurality of records and in accordance with said medical data received from said deep learning neural network.

13. The method as in claim 12, further comprising training said deep learning neural network using supervised learning that operates on labeled data so as to map a plurality of inputs to a plurality of outputs each time adjusting corresponding parameters so as to generate modified data.

14. The method as in claim 13, further comprising training said deep learning neural network using unsupervised learning that operates on unlabeled data so as to discover inherent patterns, structures, and relationships within a data set.

15. The method as in claim 14, further comprising training said deep learning neural network using forward and backward propagation through a predetermined set of hidden layers and for a plurality of epochs/iterations corresponding to a number of said hidden layers.

16. The method as in claim 11, further comprising training said deep learning neural network using data obtained in real-time from a group that includes Google Database and corresponding Data Sets, Google Scholar, Google AI Library Database, UCI machine learning repository, Disease Diagnosis Dataset libraries, Scopus, PubMed, Cleveland database, chronic kidney disease dataset, Pima diabetic dataset, Parkinsons dataset, Breast Cancer Wisconsin (Diagnostic) Data Set (WDBC dataset), Covid-chest x-ray dataset, and Web of Science (WoS).

17. The method as in claim 11, wherein said deep learning neural network is a hybrid model that includes a convolutional neural network (CNN), a recurrent neural network (RNN), multilayer perceptron, deep belief networks (DBN), and generative AI.

18. The method as in claim 11, further comprising submitting said received medical symptom data, said received diagnostic test data, said corresponding medical diagnosis and treatment options therefor as hidden layers of the deep learning neural network so as to generate a data set for submission to the Frontier Medical Literature Database framework so to transform and improve medical data stored therein.

19. The system as in claim 11, further comprising the steps of:
if the treating provider accepts that the medical diagnosis associated with said matching record should be made final, determining an insurance code associated with said accepted diagnosis associated with said matching record using the diagnostic coding database; and
displaying on a display screen said determined insurance code associated with said accepted diagnosis.

20. The system as in claim 19, wherein:
communicating said determined insurance code associated with said accepted diagnosis to a third-party coder via a wide area network;
wherein said third party coder is an insurance company; and
wherein said third party coder is a clinic or hospital.
